(19) United States
(12) Patent Application Publication
(10) Pub. No.: US 2023/0273010 A1
(43) Pub. Date: Aug. 31, 2023

(54) OPTICAL COHERENCE TOMOGRAPHY DEVICE

(71) Applicant: NEC Corporation, Minato-ku, Tokyo (JP)

(72) Inventors: Atsufumi SHIBAYAMA, Tokyo (JP); Shigeru Nakamura, Tokyo (JP)

(73) Assignee: NEC Corporation, Minato-ku, Tokyo (JP)

(21) Appl. No.: 18/019,687

(22) PCT Filed: Aug. 18, 2020
(86) PCT No.: PCT/JP2020/031114

§ 371 Date: Feb. 3, 2023

(51) Int. Cl. G01B 9/02091; G01B 9/02

(52) U.S. Cl. CPC G01B 9/02091; G01B 9/02044

(57) ABSTRACT

This optical interference tomographic imaging device includes: a wavelength-sweeping laser light source; a merged light generation means that branches the laser light into two branched lights, and outputs merged light; a branching means that branches the merged light into object light and reference light; an irradiation means that irradiates a prescribed scanning range of an object being measured with the object light; a light receiver that generates information relating to the change in intensity ratio of interference light between the reference light and the object light scattered from the object being measured; and a control means that, on the basis of the information generated by the light receiver, acquires depth-direction structure data pertaining to the object being measured.

### TECHNICAL FIELD

The this disclosure relates to an optical coherence tomography device.

### BACKGROUND ART

Techniques for performing tomography in the vicinity of the surface of a measurement target object include the optical coherence tomography (OCT) technique. In the OCT technique, tomography in the vicinity of the surface of the measurement target object is performed using interference between scattered light (hereinafter, also referred to as “backscattered light”) from the inside of the measurement target object and reference light when the measurement target object is irradiated with a light beam. In recent years, the OCT technique is increasingly applied to medical diagnosis and industrial product inspection.

In the OCT technique, a position in an optical axis direction, that is, a depth direction of a portion (light scattering point) in which object light is scattered in the measurement target object is specified using interference between the object light with which the measurement target object is irradiated and scattered and the reference light. Due to this, structure data spatially resolved in the depth direction of the measurement target object is obtained. In many cases, object light is not reflected 100% only by the surface of the measurement target object, but is propagated to the inside to some extent and then scattered backward. This makes it possible to obtain structure data spatially resolved in the depth direction inside the measurement target object. The OCT techniques include a time domain (TD-OCT) system and a Fourier domain (FD-OCT) system, and the FD-OCT system is more promising in terms of high speed and high sensitivity. In the FD-OCT system, when object light and reference light are caused to interfere with each other, an interference light spectrum in a wide wavelength band is measured and subjected to Fourier transform, and thus structure data in the depth direction is obtained. Systems for obtaining interference light spectrum include a spectral domain (SD-OCT) system using a spectrometer and a swept source (SS-OCT) system using a light source that sweeps a wavelength.

Furthermore, by scanning the measurement target object with object light in an in-plane direction perpendicular to the depth direction of the measurement target object, it is possible to obtain tomographic structure data spatially resolved in the in-plane direction and spatially resolved in the depth direction. This makes it possible to obtain three-dimensional tomographic structure data of the measurement target object. In order to irradiate different positions in the in-plane direction of the measurement target object with an object light beam, the irradiation position of one object light beam is usually scanned by a Galvano scanner or the like.

The OCT technique has been put into practical use as a tomography device for the fundus of the eye in ophthalmic diagnosis, and has been studied for application as a non-invasive tomography device for various sites of a living body.

FIG. 6 illustrates a typical configuration of an SS-OCT system optical coherence tomography device. A wavelength-swept light pulse is generated from a wavelength-swept laser light source 501. The light emitted from the wavelength-swept laser light source 501 is branched into object light R111 and reference light R121 at a branching/merging unit via a circulator 503. The object light R111 passes through a fiber collimator 505 and an irradiation optical system 506 including a scanning mirror such as a Galvano scanner and a lens, and a measurement target object 520 is irradiated with the object light R111. Then, object light R131 scattered by the measurement target object 520 returns to the branching/merging unit 504. On the other hand, the reference light R121 returns to the branching/merging unit 504 via a reference light mirror 508. Therefore, in the branching/merging unit 504, the object light R131 scattered from the measurement target object 520 and reference light R141 reflected from the reference light mirror 508 interfere with each other, and interference light R151 and interference light R161 are generated. Therefore, the intensity ratio between the interference light R151 and the interference light R161 is determined by a phase difference between the object light R131 and the reference light R141. The interference light R151 is input to a two-input balance-type light receiver 502 via the circulator 503, and the interference light R161 is directly input to the two-input balance-type light receiver 502.

The intensity ratio between the interference light R151 and the interference light R161 changes with a wavelength change of the light emitted from the wavelength-swept laser light source 501, and appears as an interference light spectrum. Therefore, the wavelength dependence of photoelectric conversion output of the balance-type light receiver 502 represents the interference light spectrum. By measuring this interference light spectrum and performing Fourier transform, it is possible to obtain data indicating the intensity of backscattered light (object light) at different positions in the depth direction (Z direction) (hereinafter, the operation of obtaining data indicating the intensity of backscattered light (object light) in the depth direction (Z direction) at a certain position of the measurement target object 520 is referred to as “A scan”).

Furthermore, the irradiation position of the object light beam R111 is scanned on the measurement target object 520 by the irradiation optical system 506. By repeatedly performing the A scan operation while moving the irradiation position of the object light beam R111 in the scanning line direction (X direction) by the irradiation optical system 506 and connecting the measurement result, it is possible to obtain, as tomographic structure data, a map of the intensity of backscattered light (object light) in two-dimension of the scanning line direction and the depth direction (hereinafter, the operation of repeatedly performing the A scan operation in the scanning line direction (X direction) and connecting the measurement result is referred to as “B scan”).

Furthermore, by repeatedly performing the B scan operation while moving the irradiation position of the object light beam R111 not only in the scanning line direction but also in the direction (Y direction) perpendicular to the scanning line by the irradiation optical system 506, and connecting the measurement result, it is possible to obtain three-dimensional tomographic structure data (hereinafter, the operation of repeatedly performing the B scan operation in the direction (Y direction) perpendicular to the scanning line and connecting the measurement results is referred to as “C scan”).

In the A scan, by acquiring an interference light spectrum with a center wavelength λ₀ and the number N of samples in a wavelength range Δλ, and performing discrete Fourier transform on the interference light spectrum, it is possible to obtain structure data in the depth direction with λ₀²/Δλ, as a unit of length. When the cycle of the A scan is ΔT and the speed in the scanning line direction of the object light beam R1 in the B scan is V, structure data (tomographic structure data) in the scanning line direction in which V×ΔT is a unit of length is obtained. That is, the positional accuracy in the three-dimensional tomographic structure data obtained by measurement by the OCT is determined depending on the operating conditions of the wavelength-swept laser light source, the Galvano scanner, and the like. Documents related to optical coherence tomography technique include PTL 1 and PTL 2.

### CITATION LIST

### Patent Literature

[PTL 1] US 2015/0363630 A
[PTL 2] US 2017/0083742 A

### SUMMARY OF INVENTION

### Technical Problem

In a case where a living body or the like is the measurement target object, it is usually difficult to completely fix and measure the measurement target object, and therefore, it is desirable to perform measurement at high speed. However, it is necessary to have a measurement time corresponding to the time required for the A scan accompanying wavelength sweep of the wavelength-swept laser light source 501 and the time required for the B scan and the C scan accompanying control of the irradiation optical system 506. If wavelength sweep is sped up, the A scan can be sped up, but speeding up the wavelength sweep is a trade-off with the characteristics of the wavelength sweep and the wideness of the wavelength sweep range, and there is a problem that high-quality, high-speed wavelength sweep is difficult. When the B scan and the C scan are sped up, the measurement accuracy decreases, and therefore speeding up has a limit.

### OBJECT OF INVENTION

An object of the this disclosure is to provide an optical coherence tomography device with a small-sized, low-cost configuration capable of performing high-speed measurement.

### Solution to Problem

In order to solve the above-described problems, an optical coherence tomography device of the this disclosure includes:
a wavelength-swept laser light source that emits laser light in an aspect of repeating an emission period in which a wavelength-swept light pulse is emitted and an interval period in which the wavelength-swept light pulse is not emitted;
a merged light generation means configured to branch the laser light into two beams of branch light, delay one of the beams of branch light by a predetermined delay time with respect to the other beam of branch light, and then merge and output, as merged light, the two beams of branch light;
a branching means configured to branch, into object light and reference light, the merged light that is incident;
an irradiation means configured to irradiate a predetermined scanning range of a measurement target object with the object light;
a light receiver that generates information regarding a change in an intensity ratio of interference light between the reference light and object light scattered from the measurement target object after the measurement target object is irradiated with the light; and
a control means configured to acquire structure data in a depth direction of the measurement target object based on information regarding a change in an intensity ratio of the interference light generated by the light receiver.

### Advantageous Effects of Invention

The optical coherence tomography device according to the this disclosure achieves high-quality, high-speed A scan, and as a result, can shorten measurement time. Since speeding up of the wavelength sweep itself and a plurality of wavelength-swept light sources are not required, a small-sized, low-cost configuration is achieved.

### BRIEF DESCRIPTION OF DRAWINGS

FIG. 1 is a view illustrating a configuration of an optical coherence tomography device according to an example embodiment of a high-level concept of the this disclosure.

FIG. 2 is a view illustrating a configuration of an optical coherence tomography device according to a first example embodiment of the this disclosure.

FIG. 3 is a waveform diagram illustrating operation of the optical coherence tomography device according to the first example embodiment of the this disclosure.

FIG. 4 is a view illustrating a configuration of an optical coherence tomography device according to a second example embodiment of the this disclosure.

FIG. 5 is a waveform diagram illustrating operation of the optical coherence tomography device according to the second example embodiment of the this disclosure.

FIG. 6 is a view illustrating a configuration of an optical coherence tomography device of a background art.

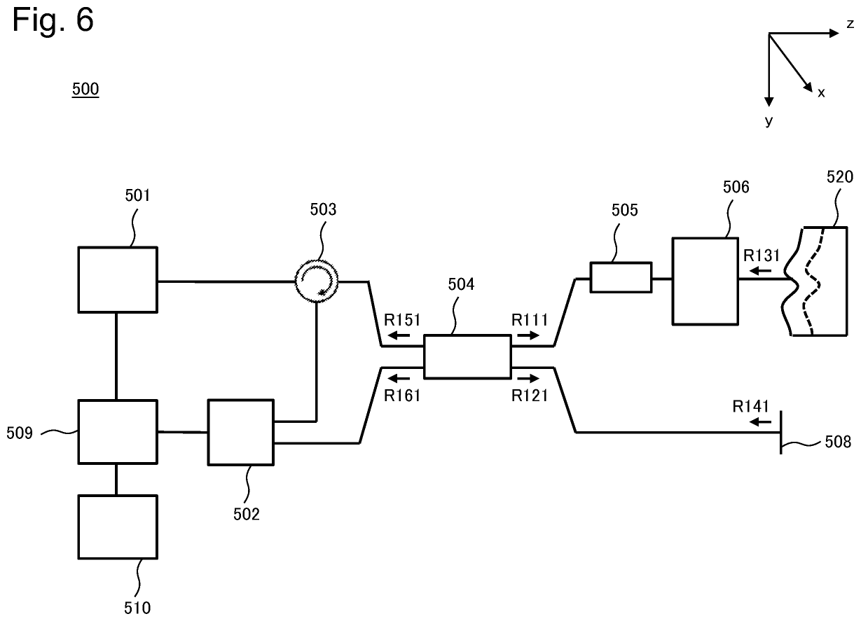

### EXAMPLE EMBODIMENT

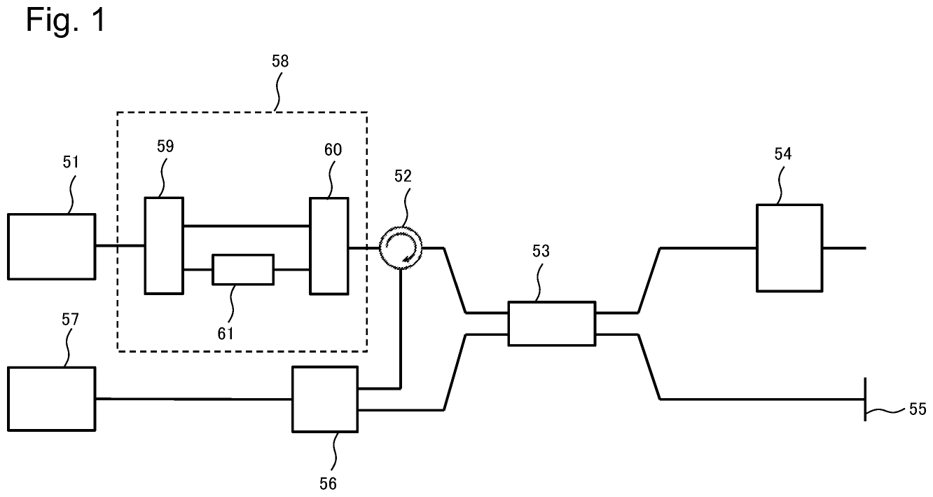

Before describing specific example embodiments, an optical coherence tomography device according to an example embodiment of a high-level concept of the this disclosure will be described. FIG. 1 is a view illustrating the configuration of an optical coherence tomography device according to an example embodiment of a high-level concept of the this disclosure. The optical coherence tomography device in FIG. 1 includes a laser light source 51, a circulator 52, an optical branching/merging unit 53, an irradiation optical system 54, a reference light mirror 55, a light receiver 56, a control unit 57, and a merged light generation unit 58. Furthermore, the merged light generation unit 58 of the optical coherence tomography device in FIG. 1 includes an optical branching unit 59, an optical merging unit 60, and a light delay unit 61.

The emission light emitted from the laser light source 51 is input to the circulator 52 via the merged light generation unit 58. The emission light emitted from the laser light source 51 is branched into two beams of emission light by the optical branching unit 59 of the merged light generation unit 58. One of the beams of emission light is directly input to the optical merging unit 60. The other of the beams of emission light becomes delay emission light delayed by a desired time by the light delay unit 66 of the merged light generation unit 58, and is input to the optical merging unit 60 of the merged light generation unit 58. The optical merging unit 60 merges the input emission light and the delay emission light, and inputs it, as merged emission light, to the optical branching/merging unit 53 via the circulator 52. The light input to the optical branching/merging unit 53 is branched into object light and reference light by the optical branching/merging unit 53.

The object light output from the optical branching/merging unit 53 passes through the irradiation optical system 54, and a measurement target object not illustrated is irradiated with the object light and scanned.

With reference to a scan control signal that is input, the irradiation optical system 54 irradiates an X-Y plane of the measurement target object with an object light beam, and scans a certain range. The object light beam with which the measurement target object is irradiated is scattered backward (in a direction opposite to the irradiation direction of the object light beam) from the measurement target object. Then, the object light (backscattered light) scattered from the measurement target object returns to the optical branching/merging unit 53 via the irradiation optical system 54. The reference light output from the optical branching/merging unit 53 is reflected by the reference light mirror 55 and returns to the optical branching/merging unit 53.

Therefore, in the optical branching/merging unit 53, the object light scattered from the measurement target object and the reference light reflected from the reference light mirror 55 interfere with each other, and two beams of interference light are obtained.

One of the beams of interference light is input to the corresponding light receiver 56 via the circulator 52, and the other of the beams of interference light is directly input to the corresponding light receiver 56. Then, interference light intensity difference information regarding the change in the intensity ratio between the two beams of interference light is generated from the light receiver 56, and interference light spectrum data is generated based on this. The control unit 57 generates two-dimensional tomographic structure data (B scan) by connecting a measurement result obtained by repeatedly performing the A scan operation while moving the irradiation position of the object light beam in the scanning line direction (at least one of the X direction and the Y direction) with respect to the measurement target object. Furthermore, the control unit 57 generates three-dimensional tomographic structure data in the X, Y, and Z directions (C scan) by connecting a measurement result obtained by repeatedly performing the B scan operation while moving the irradiation position of the object light beam in the scanning line direction and the direction perpendicular to the scanning line with respect to the measurement target object.

The emission light from the laser light source 51 in FIG. 1 is configured in an aspect of repeating an emission period in which a light pulse is emitted and an interval period in which a light pulse is not emitted. In the optical coherence tomography device of FIG. 1, the merged light generation unit 58 generates merged emission light by merging the emission light and the delay emission light where a light pulse of the emission light from the laser light source 51 is delayed so as to overlap the interval period in which the light pulse of the emission light from the laser light source 51 is not emitted.

This allows interference light spectrum data to be generated even in the interval period of emission light from the laser light source 51 where the emission period in which the light pulse is emitted and the interval period in which the light pulse is not emitted are repeated. This allows the speed of the A scan to be doubled as compared with a case where the emission light from the laser light source 51 is directly used for measurement. As a result, the measurement time can be reduced to ½. Hereinafter, more specific example embodiments will be described.

### First Example Embodiment

Hereinafter, example embodiments of the this disclosure will be described with reference to the drawings.

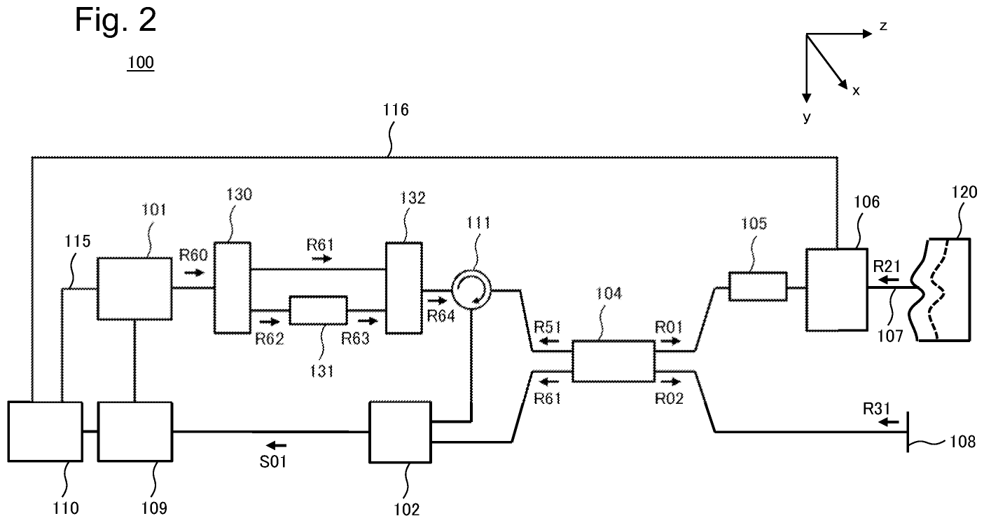

An optical coherence tomography device 100 according to the first example embodiment of the this disclosure will be described. FIG. 2 is a view illustrating an example of the optical coherence tomography device according to the first example embodiment. As illustrated in FIG. 2, the optical coherence tomography device 100 includes a wavelength-swept laser light source 101, a circulator 111, an optical branching/merging unit 104, a fiber collimator 105, an irradiation optical system 106, a reference light mirror 108, a balance-type light receiver 102, a light spectrum data generation unit 109, and a control unit 110. Furthermore, the optical coherence tomography device 100 of FIG. 2 includes an optical branching unit 130, a light delay unit 131, and an optical merging unit 132.

The wavelength-swept laser light source 101 generates a wavelength-swept light pulse.

Emission light R60 emitted from the wavelength-swept laser light source 101 is branched into emission light R61 and emission light R62 by the optical branching unit 130. The emission light R61 is directly input to the optical merging unit 132. The emission light R62 is input to the light delay unit 131. The light delay unit 131 delays the input emission light R62 by a desired time and inputs it, as delay emission light R63, to the optical merging unit 132. The optical merging unit 132 merges the input emission light R61 and the delay emission light R63, and inputs it, as merged emission light R64, to the optical branching/merging unit 104 via the circulator 111. The light input to the optical branching/merging unit 104 is branched into object light R01 and reference light R02 by the optical branching/merging unit 104.

The object light R01 output from the optical branching/merging unit 104 passes through the fiber collimator 105 and the irradiation optical system 106, and a measurement target object 120 is irradiated with the object light and scanned.

With reference to a scan control signal 116 given from the control unit 110, the irradiation optical system 106 irradiates different positions on an X-Y plane of the measurement target object 120 with an object light beam 107, and scans a certain range.

The object light beam 107 with which the measurement target object 120 is irradiated is scattered backward (in a direction opposite to the irradiation direction of the object light beam) from the measurement target object 120. Then, object light (backscattered light) R21 scattered from the measurement target object 120 returns to the optical branching/merging unit 104 via the irradiation optical system 106 and the fiber collimator 105.

The reference light R02 output from the optical branching/merging unit 104 is reflected by the reference light mirror 108 and returns to the optical branching/merging unit 104.

Therefore, in the optical branching/merging unit 104, the object light R21 scattered from the measurement target object 120 and reference light R31 reflected from the reference light mirror 108 interfere with each other, and interference light R51 and interference light R61 are obtained.

The interference light R51 is input to the corresponding balance-type light receiver 102 via the circulator 111, and the interference light R61 is directly input to the corresponding balance-type light receiver 102. Then, interference light intensity difference information S01 regarding the change in the intensity ratio between the interference light R51 and the interference light R61 is input from the balance-type light receiver 102 to the light spectrum data generation unit 109. Note that the balance-type light receiver 102 is a light receiver having a configuration in which two photodiodes are connected in series and the connection is the output (differential output).

Here, interference between object light having a wavelength λ, and a wavenumber k (=2π/λ) and reference light is considered. In a case where the optical path length from when the reference light is branched by the optical branching/merging unit 104 to when the reference light is reflected by the reference light mirror 108 and returns to the optical branching/merging unit 104 is LR and the optical path length from when the object light is branched by the optical branching/merging unit 104 to when the object light is backscattered at one light scattering point of the measurement target object 120 and returns to the optical branching/merging unit 104 is LS=LR+z₀, the object light R21 and the reference light R31 interfering at the optical branching/merging unit 104 interfere with each other at a phase difference kz₀+φ. Here, φ is a constant that does not depend on k or z₀. Let the amplitude of the object light R21 interfering at the optical branching/merging unit 104 be Eₛ and let the amplitude of the reference light R31 be E_{R}, the intensity difference between the interference light R51 and the interference light R61 represented by

I(k)∝E_{S}·E_{R}·cos(kz₀+ϕ) [Math. 1]

is photoelectrically converted by the balance-type light receiver 102. The light spectrum data generation unit 109 generates interference light spectrum data based on the information regarding the wavelength change of the emission light from the wavelength-swept laser light source and the interference light intensity difference information S01 regarding the intensity difference between the interference light R51 and the interference light R61 from the balance-type light receiver 102. Modulation with the cycle 2π/z₀ appears in interference light spectrum data I(k) obtained by measuring from the wavenumbers k₀−Δk/2 to k₀+Δk/2. The obtained interference light spectrum data is sent from the light spectrum data generation unit 109 to the control unit 110.

The control unit 110 performs Fourier transform on the interference light spectrum data. Amplitude J(z) of the Fourier transform of I(k) becomes

□(□)=|∫□(□)□^{□□□}|∝□(□−□₀)+□(□+□₀) [Math. 2]

and a δ function peak is shown at z=z₀ (and z=−z₀) reflecting a light scattering point position z₀. A mirror has one light scattering point position, but usually, the object light with which the measurement target object is irradiated is sequentially backscattered while being attenuated and propagated to the inside to some extent, and the light scattering point of the object light is distributed in a range from the surface to a certain depth. In a case where the light scattering point is distributed from z₀−Δz to z₀+Δz in the depth direction, modulation from the cycles 2π/(z₀−Δz) to 2π/(z₀+Δz) appears in an overlapping manner in the interference light spectrum.

The control unit 110 controls each unit of the optical coherence tomography device 100.

The control unit 110 controls the irradiation optical system 106 so as to irradiate different positions on the X-Y plane of the measurement target object 120 with the object light beam 107. The control unit 110 controls the cycle and speed at which the irradiation optical system 106 scans the measurement target object 120.

The control unit 110 generates two-dimensional tomographic structure data (B scan) by connecting a measurement result obtained by repeatedly performing the A scan operation while moving the irradiation position of the object light beam 107 in the scanning line direction (at least one of the X direction and the Y direction).

The control unit 110 generates three-dimensional tomographic structure data in the X, Y, and Z directions (C scan) by connecting a measurement result obtained by repeatedly performing the B scan operation while moving the irradiation position of the object light beam in the scanning line direction and the direction perpendicular to the scanning line.

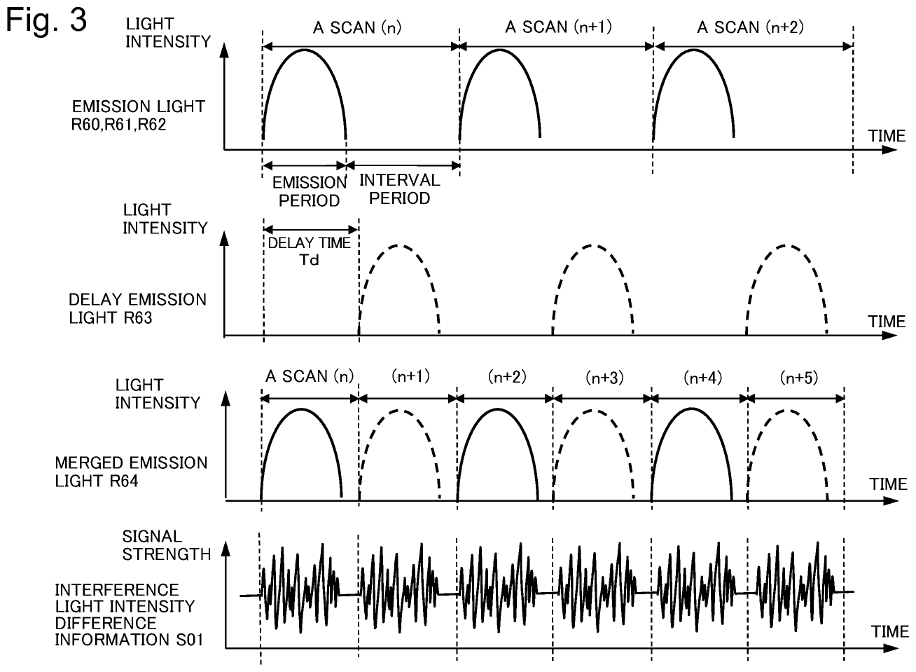

Next, the A scan operation in the present example embodiment will be described in detail with reference to FIG. 3. FIG. 3 is a waveform diagram illustrating an example of the emission light R60, the emission light R61, the emission light R62, the delay emission light R63, the merged emission light R64, and the interference light intensity difference information S01.

The emission light R60 emitted from the wavelength-swept laser light source 101 is configured by repeating an emission period in which a wavelength-swept light pulse is actually emitted and an interval period in which a light pulse is not emitted. A scan (n) in FIG. 3 represents n-th A scan, A scan (n+1) represents (n+1)-th A scan to be performed next to the nth A scan, and A scan (n+2) represents (n+2)-th A scan to be performed next to the (n+1)-th A scan. Consecutive one emission period and one interval period correspond to one A scan operation. Therefore, in order to speed up the A scan, it is desirable that the emission period and the interval period be short, but the emission period depends on the range of wavelength sweep and the characteristics of sweep, and is not easily shortened. The interval period is a necessary period that is secured for reasons such as preparation for the next wavelength sweep and inability to generate a light pulse that can be used for measurement in wavelength sweep operation, and is similarly not easily shortened.

In the present example embodiment, a case where the interval period is longer than the emission period will be described.

In FIG. 3, the light delay unit 131 of the present example embodiment generates the delay emission light R63 in which the emission light R62 is delayed by a predetermined time so that the emission period of the emission light R62 is provided within the range of the interval period of the delay emission light R61. In other words, the delay emission light R63 is light in which the emission light R62 is delayed by a predetermined time such that the emission period of the emission light R62 is provided within the range of the interval period of the delay emission light R61 by the light delay unit 131. Specifically, the delay emission light R63 is light in which the emission light R62 is delayed by delay time Td satisfying Tp≤Td≤Ti, where the length of the emission period is Tp and the length of the interval period is Ti.

The optical merging unit 132 merges the emission light R61 and the delay emission light R63 to generate the merged emission light R64. In other words, the merged emission light R64 is light in which the emission light R61 and the delay emission light R63 are merged by the optical merging unit 132.

The interference light intensity difference information S01 is information regarding a change in the intensity ratio between the interference light R51 and the interference light R61 that are generated by the balance-type light receiver 102.

The light spectrum data generation unit 109 generates interference light spectrum data based on the information regarding the wavelength change of the emission light from the wavelength-swept laser light source and the interference light intensity difference information S01.

A specific numerical example of the delay time Td will be described below.

As an example, the wavelength-swept laser light source 101 emits a wavelength-swept light pulse with an emission period length of Tp=4 μs and an interval period length of Ti=6 μs, that is, with a cycle of Tp+Ti=10 μs. The light delay unit 131 delays the emission light R62 by Td=5 μs as a delay time satisfying Tp≤Td≤Ti, that is, 4 μs≤Td≤6 μs. For example, in a case where the light delay unit 131 includes an optical fiber having a light propagation speed of 200,000 km/sec, the length of the optical fiber becomes 1 km.

### Effects of First Example Embodiment

As described above, the optical coherence tomography device 100 according to the first example embodiment provides the wavelength-swept light pulse also in the interval period in which the wavelength-swept light pulse is not emitted in the emission light R60 emitted from the wavelength-swept laser light source 101.

Therefore, interference light spectrum data can be generated even in the interval period of the emission light R60 in such a manner of repeating the emission period in which a wavelength-swept light pulse is actually emitted and the interval period in which a light pulse is not emitted. This allows the speed of the A scan to be doubled as compared with a case where the emission light R60 from the wavelength-swept laser light source 101 is directly used for measurement. As a result, the measurement time can be reduced to ½.

For example, in the range illustrated in FIG. 3, when the emission light R60 is used directly for measurement, three times of A scan from the A scan (n) to the A scan (n+2) are performed, whereas when the merged emission light R64 of the present example embodiment is used for measurement, six times of A scan from the A scan (n) to A scan (n+5) can be performed.

Since the wavelength-swept laser light source 101 itself can set an appropriate emission period and interval period that can achieve desired wavelength sweeping characteristics and device size, a small-sized, low-cost wavelength-swept laser light source can be achieved. A plurality of wavelength-swept laser light sources are not required, and as a result, a small-sized, low-cost optical coherence tomography device can be achieved.

### Second Example Embodiment

Next, an optical coherence tomography device 200 according to the second example embodiment of the this disclosure will be described.

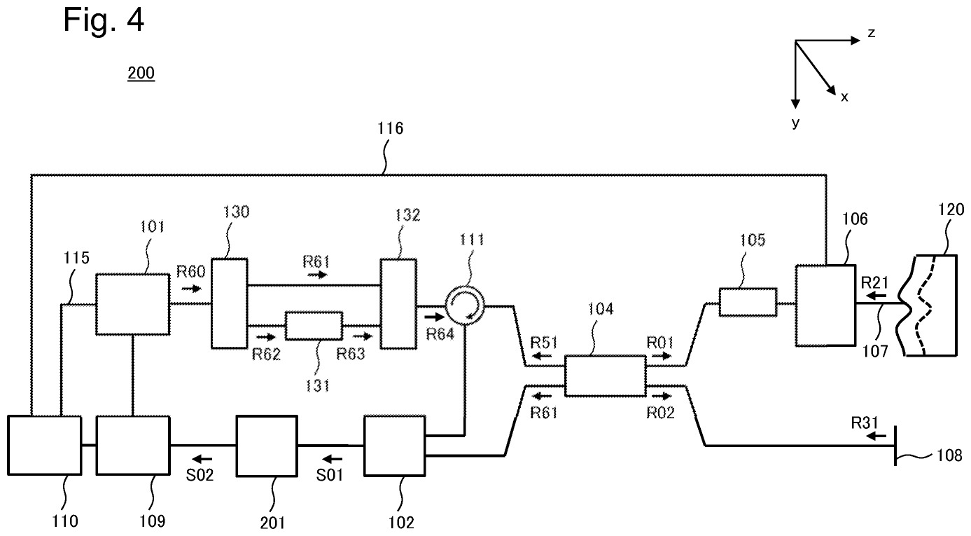

FIG. 4 is a view illustrating an example of the optical coherence tomography device 200 according to the second example embodiment.

In FIG. 4, the optical coherence tomography device 200 has a configuration in which an interference light intensity difference information selection unit 201 is added to the optical coherence tomography device 100 according to the first example embodiment, and the same components as those of the optical coherence tomography device are denoted by the same reference numerals, and the detailed description will be omitted below.

As illustrated in FIG. 4, the optical coherence tomography device 200 includes the wavelength-swept laser light source 101, the circulator 111, the optical branching/merging unit 104, the fiber collimator 105, the irradiation optical system 106, the reference light mirror 108, the balance-type light receiver 102, the light spectrum data generation unit 109, and the control unit 110. Furthermore, the optical coherence tomography device 200 of FIG. 4 includes the optical branching unit 130, the light delay unit 131, and the optical merging unit 132, similarly to the first example embodiment. Furthermore, the optical coherence tomography device 200 of FIG. 4 includes the interference light intensity difference information selection unit 201.

The interference light intensity difference information selection unit 201 of the present example embodiment generates interference light intensity difference information S02 in which a part of the interference light intensity difference information S01 is selected, based on the information regarding the wavelength change of the emission light from the wavelength-swept laser light source 101 and the interference light intensity difference information S01 regarding the intensity difference between the interference light R51 and the interference light R61 from the balance-type light receiver 102. The light spectrum data generation unit generates interference light spectrum data based on the information regarding the wavelength change of the emission light from the wavelength-swept laser light source 101 and the interference light intensity difference information S02 from the interference light intensity difference information selection unit 201.

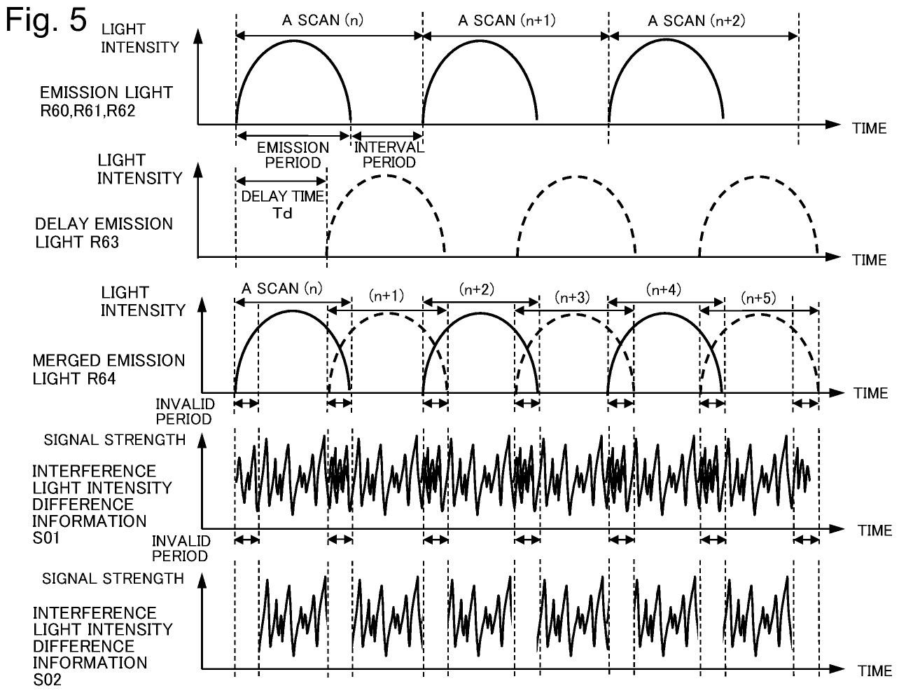

Next, the A scan operation in the present example embodiment will be described in detail with reference to FIG. 5. FIG. 5 is a waveform diagram illustrating an example of the emission light R60, the emission light R61, the emission light R62, the delay emission light R63, the merged emission light R64, the interference light intensity difference information S01, and the interference light intensity difference information S02. In the present example embodiment, a case where the interval period is shorter than the emission period will be described.

In FIG. 5, the light delay unit 131 of the present example embodiment generates the delay emission light R63 in which the emission light R62 is delayed by a predetermined time so that the emission period of the emission light R62 is provided within the range of the interval period of the delay emission light R61. In other words, the delay emission light R63 is light in which the emission light R62 is delayed by a predetermined time such that the interval period of the delay emission light R61 is provided within the range of the emission period of the emission light R62 by the light delay unit 131. Specifically, it is light in which the emission light R62 is delayed by delay time Td satisfying Ti≤Td≤Tp, where the length of the emission period is Tp and the length of the interval period is Ti.

The optical merging unit 132 merges the emission light R61 and the delay emission light R63 to generate the merged emission light R64. In other words, the merged emission light R64 is light in which the emission light R61 and the delay emission light R63 are merged by the optical merging unit 132.

The interference light intensity difference information S01 is information regarding a change in the intensity ratio between the interference light R51 and the interference light R61 that are generated by the balance-type light receiver 102.

The interference light intensity difference information S02 is a signal generated by selecting a part having valid information from the interference light intensity difference information S01 by the interference light intensity difference information selection unit 201. Specifically, it is a signal in which in the merged emission light R64, a part of the invalid period of the interference light intensity difference information S01 corresponding to the invalid period that is a period in which the emission period of the emission light R61 and the emission period of the delay emission light R63 overlap each other is removed, and a valid period other than the invalid period is selected.

That is, the interference light intensity difference information selection unit 201 provides a function of removing, from the interference light intensity difference information S01, an invalid period that is a period in which valid measurement data is not obtained due to beams of light of different wavelengths being mixed, and selecting only a period in which valid measurement data is obtained. The invalid period and the valid period can be specified from the information regarding the wavelength change of the emission light from the wavelength-swept laser light source 101 and the delay time Td. Specifically, when the start time of the emission period that can be specified from the information regarding the wavelength change of the emission light is tn, a period from time tn to tn+(Tp−Td) and a period from time tn+Td to tn+Tp are invalid periods, and the other periods from time tn+(Tp−Td) to time tn+Td are valid periods.

The light spectrum data generation unit 109 generates interference light spectrum data based on the information regarding the wavelength change of the emission light from the wavelength-swept laser light source and the interference light intensity difference information S02.

A specific numerical example in the present example embodiment will be described below.

As an example, the wavelength-swept laser light source 101 emits a wavelength-swept light pulse with an emission period length of Tp=6 μs and an interval period length of Ti=4 μs, that is, with a cycle of Tp+Ti=10 μs. The light delay unit 131 delays the emission light R62 by Td=5 μs as a delay time satisfying Ti≤Td≤Tp, that is, 4 μs≤Td≤6 μs.

In the interference light intensity difference information selection unit 201, when the start time of the emission period is tn, a period of 1 μs in length from time tn to tn+1 μs, which corresponds to a period from time tn to tn+(Tp−Td), and a period of 1 μs in length from time tn+5 μs to tn+6 μs, which corresponds to a period from time tn+Td to tn+Tp, are removed as invalid periods, and a period of 4 μs in length from time tn+1 μs to tn+5 μs, which corresponds to the other period from time tn+(Tp−Td) to tn+Td, is selected as a valid period. That is, in the emission period of 6 μs, the period of 4 μs excluding the invalid period of 2 μs in total becomes the valid period in which valid interference light spectrum data is obtained.

### Effects of Second Example Embodiment

As described above, similarly to the first example embodiment, the optical coherence tomography device 200 according to the second example embodiment provides the wavelength-swept light pulse also in the interval period in which the wavelength-swept light pulse is not emitted in the emission light R60 emitted from the wavelength-swept laser light source 101.

Therefore, interference light spectrum data can be generated even in the interval period of the emission light R60 in such a manner of repeating the emission period in which a wavelength-swept light pulse is actually emitted and the interval period in which a light pulse is not emitted. The optical coherence tomography device 200 according to the second example embodiment can generate interference light spectrum data in the interval period even when the interval period is shorter than the emission period different from that of the first example embodiment. This allows the speed of the A scan to be doubled as compared with a case where the emission light R60 from the wavelength-swept laser light source 101 is directly used for measurement. As a result, the measurement time can be reduced to ½. For example, in the range illustrated in FIG. 5, when the emission light R60 is used directly for measurement, three times of A scan from the A scan (n) to the A scan (n+2) are performed, whereas when the merged emission light R64 of the present example embodiment is used for measurement, six times of A scan from the A scan (n) to A scan (n+5) can be performed.

On the other hand, in the present example embodiment, the period in which valid interference light spectrum data is obtained becomes shorter than the emission period, and the range of wavelength sweep is reduced accordingly. As a result, the measurement accuracy in the depth direction (Z direction) decreases, but the present example embodiment is valid in a case where the required measurement accuracy can be obtained even in that case or in a case where the measurement speed is prioritized over the measurement accuracy. Similarly to the first example embodiment, since the wavelength-swept laser light source 101 itself can set an appropriate emission period and interval period that can achieve desired wavelength sweeping characteristics and device size, a small-sized, low-cost wavelength-swept laser light source can be achieved. A plurality of wavelength-swept laser light sources are not required, and as a result, a small-sized, low-cost optical coherence tomography device can be achieved.

The this disclosure has been described above using the above-described example embodiments as exemplary examples. However, the this disclosure is not limited to the above-described example embodiments. That is, the this disclosure can be applied with various aspects that can be understood by those of ordinary skill in the art within the scope of the this disclosure.

### REFERENCE SIGNS LIST

100 optical coherence tomography device
101 wavelength-swept laser light source
102 balance-type light receiver
104 optical branching/merging unit
105 fiber collimator
106 irradiation optical system
107 object light beam
108 reference light mirror
109 light spectrum data generation unit
110 control unit
115 wavelength-swept control signal
116 scan control signal
111 circulator
120 measurement target object
130 optical branching unit
131 light delay unit
132 optical merging unit
201 interference light intensity difference information selection unit
500 optical coherence tomography device
501 wavelength-swept laser light source
502 balance-type light receiver
504 branching/merging unit
505 fiber collimator
506 irradiation optical system
507 object light beam
508 reference light mirror
509 light spectrum data generation unit
510 control unit
511 circulator
520 measurement target object

What is claimed is:

1. An optical coherence tomography device comprising:
a wavelength-swept laser light source that emits laser light in an aspect of repeating an emission period in which a wavelength-swept light pulse is emitted and an interval period in which the wavelength-swept light pulse is not emitted;
a light generator configured to branch the laser light into two beams of branch light, delay one of the beams of branch light by a predetermined delay time with respect to another beam of branch light, and then merge and output, as merged light, the two beams of branch light;
a branching unit configured to branch, into object light and reference light, the merged light that is incident;
an irradiator configured to irradiate a predetermined scanning range of a measurement target object with the object light;
a light receiver that generates information regarding a change in an intensity ratio of interference light between the reference light and object light scattered from the measurement target object after the measurement target object is irradiated with the light; and
a controller configured to acquire structure data in a depth direction of the measurement target object based on information regarding a change in an intensity ratio of the interference light generated by the light receiver.

2. The optical coherence tomography device according to claim 1, wherein
the delay time is longer than a length of the emission period and shorter than a sum of a length of the emission period and a length of the interval period.

3. The optical coherence tomography device according to claim 1, wherein
the delay time is equal to or more than a length of the emission period and equal to or less than a length of the interval period.

4. The optical coherence tomography device according to claim 1, wherein
the delay time is equal to or more than a length of the interval period and equal to or less than a length of the emission period.

5. The optical coherence tomography device according to claim 4 further comprising:
a selector configured to generate second information regarding wavelength dependency of interference light intensity by selecting a part including valid measurement data from first information regarding a change in an intensity ratio of the interference light generated by the light receiver.

6. The optical coherence tomography device according to claim 5, wherein
the second information is
information corresponding to a period in which a period in which the wavelength-swept light pulse of the one branch light delayed by the predetermined delay time is emitted and a period in which the wavelength-swept light pulse of the other branch light is emitted do not overlap each other.

7. The optical coherence tomography device according to claim 6, wherein
the selector generates the second information by selecting, in the merged light, a time period tn+(Tp−Td) to tn+Td, wherein tn is time at which the wavelength-swept light pulse is emitted, a length Tp is the emission period, and Td is the delay time.
